(19) United States
(12) Patent Application Publication
(10) Pub. No.: US 2026/0053397 A1
(43) Pub. Date: Feb. 26, 2026

(54) DIET RECOMMENDATIONS USING CHEMICAL SENSORS

(71) Applicant: Cardiac Pacemakers, Inc., St Paul, MN (US)

(72) Inventors: Pramodsingh Hirasingh Thakur, Woodbury, MN (US); Michael J. Kane, St Paul, MN (US); Ramesh Wariar, Blaine, MN (US); Yingbo Li, Woodbury, MN (US)

(21) Appl. No.: 19/303,695

(22) Filed: Aug. 19, 2025

Related U.S. Application Data
(60) Provisional application No. 63/685,253, filed on Aug. 20, 2024.

(51) Int. Cl. A61B 5/145; A61B 5/00; A61B 5/1455; A61B 5/1473

(52) U.S. Cl. CPC A61B 5/14546; A61B 5/14514; A61B 5/1455; A61B 5/14735; A61B 5/6898; A61B 5/7271; A61B 5/742; A61B 2560/0462

(57) ABSTRACT

Systems, devices, and methods include approaches involving comparing an analyte level measured using a chemical sensor and based on an optical property to a threshold; determining that a creatinine level, a potassium level, or a sodium has reached the threshold; and generating a visual recommendation on a user interface to consume a source of protein, a source of potassium, or a source of sodium.

### CROSS REFERENCE TO RELATED APPLICATION

This application claims priority to Provisional Application No. 63/685,253, filed Aug. 20, 2024, which is herein incorporated by reference in its entirety.

### TECHNICAL FIELD

Instances of the present disclosure relate to using analyte sensing technology for evaluating and recommending diets.

### BACKGROUND

Diet recommendations can improve analyte levels of patients experiencing health issues.

### SUMMARY

In Example 1, a method includes comparing an analyte level measured using a chemical sensor (e.g., based on an optical property of the chemical sensor) to a threshold. In certain instances, the analyte level is a creatinine level, a potassium level, or a sodium. The method further includes determining that the creatinine level, the potassium level, or the sodium level has reached the threshold. After the determining, the method includes generating a visual recommendation on a user interface to consume or refrain from consuming a source of protein, a source of potassium, or a source of sodium.

In Example 2, the method of Example 1, wherein the chemical sensor is a wearable device that includes needles sized for access to interstitial fluid and a chemical indicator positioned within the needles.

In Example 3, the method of Example 2, wherein the chemical indicator changes the optical property in response to different creatinine levels, potassium levels, or sodium levels.

In Example 4, the method of any of Examples 1-3, wherein the user interface is part of a mobile computing device that includes an image sensor.

In Example 5, the method of Example 4, further including: determining the creatinine level, the potassium level, or the sodium based, at least in part, on the optical property contain in a digital image taken by the image sensor.

In Example 6, the method of any of Examples 1-5, wherein the visual recommendation displays an amount or a range of the source.

In Example 7, the method of any of Examples 1-6, wherein the visual recommendation displays a type of source.

In Example 8, the method of any of Examples 1-7, further including: generating the visual recommendation only if the creatine level, the potassium level, or the sodium level is below a minimum level.

In Example 9, the method of any of Examples 1-8, wherein the threshold is a predetermined percentage below a predetermined level or below historical analyte data.

In Example 10, the method of any of Examples 1-9, wherein the threshold is a predetermined level below a predetermined level or below historical analyte data.

In Example 11, the method of any of Examples 1-10, wherein the analyte level is the creatinine level, wherein the creatinine level is a serum concentration of creatinine.

In Example 12, the method of any of Examples 1-10, wherein the analyte level is the potassium level.

In Example 13, a computer program product comprising instructions to cause one or more processors to carry out the steps of the method of Examples 1-12.

In Example 14, a computer-readable medium having stored thereon the computer program product of Example 13.

In Example 15, a mobile device comprising the computer-readable medium of Example 14.

In Example 16, a system includes a mobile computing device with a processor, memory, and a user interface. The mobile computing device is programmed to compare an analyte level measured using a chemical sensor (e.g., based on an optical property) to a threshold. The analyte level is a creatinine level, a potassium level, or a sodium level. The mobile computing device is further programmed to determine that the creatinine level, the potassium level, or the sodium has reached the threshold and—after the determining-display a visual recommendation on the user interface to consume a source of protein, a source of potassium, or a source of sodium.

In Example 17, the system of Example 16, further including: the chemical sensor, wherein the chemical sensor is a wearable device with a first set of needles sized for access to interstitial fluid and a first chemical indicator positioned within the first set of needles, wherein the first chemical indicator changes optical properties in response to different creatinine levels.

In Example 18, the system of Example 17, wherein the chemical sensor includes a second set of needles sized for access to interstitial fluid and a second chemical indicator positioned within the second set of needles, wherein the second chemical indicator changes optical properties in response to different potassium levels.

In Example 19, the system of Example 17, wherein the chemical sensor includes a second set of needles sized for access to interstitial fluid and a second chemical indicator positioned within the second set of needles, wherein the second chemical indicator changes optical properties in response to different sodium levels.

In Example 20, the system of Example 17, wherein the mobile computing device includes an image sensor.

In Example 21, the system of Example 20, wherein the mobile computing device is programmed to: determine the creatinine level based, at least in part, on an optical property of a chemical indicator.

In Example 22, the system of Example 16, wherein the mobile computing device includes an image sensor and is programmed to: determine the creatinine level, the potassium level, or the sodium level based, at least in part, on an optical property of the chemical indicator in a digital image.

In Example 23, the system of Example 16, further including: the chemical sensor, wherein the chemical sensor is part of an implantable medical device and comprises a chemical indicator, wherein the chemical indicator changes optical properties in response to different creatine levels, potassium levels, or sodium levels.

In Example 24, the system of Example 16, wherein the visual recommendation displays an amount or a range of the source.

In Example 25, the system of Example 16, wherein the visual recommendation displays a type of source.

In Example 26, the system of Example 16, wherein the mobile computing device is programmed to: generate the visual recommendation only if the creatine level, the potassium level, or the sodium level is below a minimum level.

In Example 27, the system of Example 16, wherein the threshold is a predetermined percentage below a predetermined level or below historical analyte data.

In Example 28, the system of Example 16, wherein the threshold is a predetermined level below a predetermined level or below historical analyte data.

In Example 29, a method includes comparing an analyte level measured using a chemical sensor (e.g., based on an optical property) to a threshold. The analyte level is a creatinine level, a potassium level, or a sodium level. The method further includes determining that the creatinine level, the potassium level, or the sodium level has reached the threshold and—after the determining—generating a visual recommendation on a user interface to consume or refrain from consuming a source of protein, a source of potassium, or a source of sodium.

In Example 30, the method of Example 29, further including: determining the creatinine level, the potassium level, or the sodium level based, at least in part, on the optical property contained in a digital image taken by an image sensor.

In Example 31, the method of Example 29, wherein the visual recommendation displays an amount or a range of the source.

In Example 32, the method of Example 29, wherein the visual recommendation displays a type of source.

In Example 33, the method of Example 29, further including: generating the visual recommendation only if the creatine level, the potassium level, or the sodium level is below a minimum level.

In Example 34, the method of Example 29, wherein the threshold is a predetermined percentage below a predetermined level or below historical analyte data.

In Example 35, the method of Example 29, wherein the threshold is a predetermined level below a predetermined level or below historical analyte data.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 is a schematic illustration of a chemical sensing system, in accordance with certain instances of the present disclosure.

FIG. 2 shows a block diagram of a method for use with one or more components of a chemical sensing system, in accordance with certain instances of the present disclosure.

FIG. 3 shows a schematic illustration of a computing device, in accordance with certain instances of the present disclosure.

FIG. 4 shows a graph of a person's analyte response to consuming food, in accordance with certain instances of the present disclosure.

FIG. 5 shows a block diagram of a method for use with one or more components of a chemical sensing system, in accordance with certain instances of the present disclosure.

FIGS. 6-8 show different views of various portions of a wearable chemical sensing device, in accordance with certain instances of the present disclosure.

FIGS. 9 and 10 show different views of various portions of an implantable medical device, in accordance with certain instances of the present disclosure.

FIG. 11 shows a block diagram with additional details of the computing device of FIG. 3, in accordance with certain instances of the present disclosure.

While the disclosed subject matter is amenable to various modifications and alternative forms, specific instances have been shown by way of example in the drawings and are described in detail below. The intention, however, is not to limit the disclosed subject matter to the particular instances described. On the contrary, the disclosed subject matter is intended to cover all modifications, equivalents, and alternatives falling within the scope of the disclosed subject matter as defined by the appended claims.

### DETAILED DESCRIPTION

Certain analytes (e.g., creatinine, potassium, sodium) can be measured and monitored to evaluate kidney and/or cardiac conditions and performance.

One example analyte is creatinine, which is an indicator of a person's renal function. Creatinine is created as a byproduct of a muscle mass breakdown (e.g., muscle metabolism), and a person's kidneys are supposed to filter (e.g., excrete) creatinine from the bloodstream. When renal function declines, less creatinine is excreted from the body, and serum concentrations of creatinine rise. Also, a person's renal function depends on adequate cardiac output, so a declining renal function indicates a decline in cardiac output. As such, measuring and monitoring creatinine levels can be used to evaluate whether a person's kidneys and heart are functioning properly.

Eating protein (e.g., foods containing protein) can increase the amount of creatinine a person produces and therefore cause their kidneys to work harder to attempt to filter out the creatinine. As such, it is generally recommended that a person with failing kidneys reduce their protein consumption. However, consuming less protein can result in less muscle mass, which can be problematic for maintaining musculoskeletal health. Certain instances of the present disclosure are accordingly directed to approaches (e.g., systems, methods, devices) that use analyte sensing technology for evaluating analyte levels (e.g., creatinine levels) and making diet recommendations such as timing, composition, amounts, and sources for protein consumption.

Another example analyte is potassium. Similar to creatinine, a person's kidneys are supposed to filter potassium from the bloodstream (although a typical person's kidneys only filter approximately 90% of potassium). The excitation cycle of cardiac cells is influenced by the cardiac cells' resting electrical potential and by the activity of ion channels (such as potassium, sodium, and calcium ion channels) in the cell membrane of the cardiac cells. When the concentration of potassium in plasma is within a normal range, the potassium ion channels can function effectively. However, when the potassium concentration in the plasma is elevated (referred to as “hyperkalemia”), the concentration gradient of potassium across the cardiac cell membrane is reduced and the cardiac cell generally becomes depolarized and inexcitable. In contrast, when the potassium concentration is low (referred to as “hypokalemia”), the concentration gradient of potassium across the cardiac cell membrane is increased resulting in hyperpolarization of the resting electrical potential. Hypokalemia and hyperkalemia can lead to arrhythmias, such as atrial fibrillation. As such, measuring and monitoring potassium levels can be used to evaluate whether a person's heart is functioning properly.

Certain instances of the present disclosure are accordingly directed to approaches (e.g., systems, methods, devices) that use analyte sensing technology for evaluating analyte levels and making diet recommendations such as timing, composition, amounts, and types of potassium consumption.

The approaches described herein can also be used to monitor and evaluate other analytes such as sodium.

### Analyte Sensing System

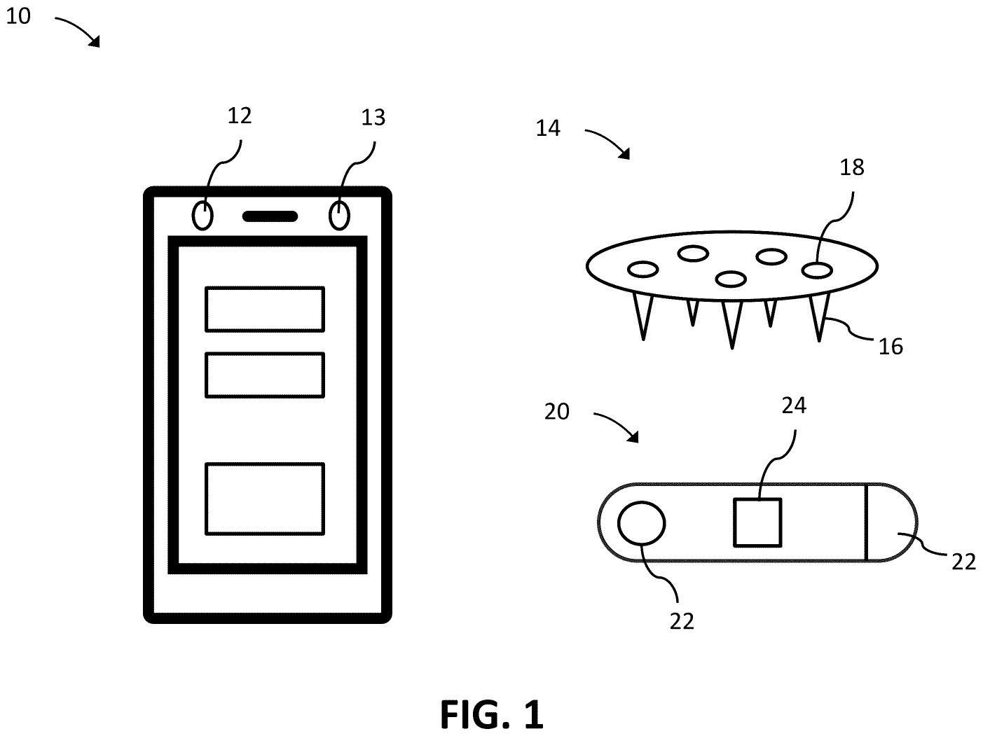

FIG. 1 shows a chemical sensing system 10 (hereinafter “the system 10” for brevity) with schematic representations of two sets of components that can be used to measure analyte concentrations. Typically, measuring a patient's analyte concentrations requires drawing multiple blood samples from a patient at a clinic and processing the blood samples at a laboratory. Components of the chemical sensing system 10 can be used to measure analyte concentrations using a wearable device or an implantable medical device. Although the chemical sensors shown in FIG. 1 and described below are optical-based chemical sensors, other types of chemical sensor approaches can be used (e.g., electrical-based chemical sensors such as those that use electrodes and measure potential differences, and the like).

For the wearable approach, the system 10 can include a device (e.g., a mobile computing device described further herein) with an image sensor 12 and a chemical sensing device 14. The image sensor 12 (e.g., a charge coupled device, a complementary metal oxide semiconductor, or other devices that can capture an image) can be part of a camera, smart phone, or other device able to capture an image (e.g., a digital image). In certain instances, the image sensor 12 and the chemical sensing device 14 are integrated into a single device, and in other instances the image sensor 12 and the chemical sensing device 14 are separate devices. In instances where the image sensor 12 is part of a mobile computing device such as a smart phone, the smart phone can store, operate, or otherwise access a program (e.g., a phone application) that processes an image (of the chemical sensing device 14) taken by the image sensor 12 and determines estimates of one or more analyte concentrations of the patient. In other instances, the image sensor 12 is part of a dedicated readout device or part of a camera. The system 10 can include one or more light sources 13, which can be part of the same device as the image sensor 12 or which can be part of a separate component. The one or more light sources 13 can generate light (e.g., emit visible light, ultraviolet light, monochromatic light (red, green, blue)).

The chemical sensing device 14 can be a wearable device (e.g., an exterior device and not an implantable device) such as a device that includes (or is part of) a strap (e.g., an armband strap), a patch (e.g., a torso patch), or another type of device that can be coupled to a patient's skin. For simplicity, the chemical sensing device 14 is hereinafter referred to as the “patch 14” although other types of wearable devices can use the chemical sensing technology described herein.

In certain instances, the patch 14 is a transdermal patch that includes a mechanism (e.g., needles 16) that provides access to a patient's interstitial fluid. For example, multiple needles 16 (e.g., microneedles) can be sized to access a patient's interstitial fluid. The patch 14 can also include multiple chemical indicators 18, each of which changes optical properties (e.g., fluorimetric properties, colorimetric properties) with changes in concentration of a certain analyte in the interstitial fluid. As described in more detail herein, the image sensor 12 can be used to capture an image (e.g., a digital image) of the chemical indicators 18, and the image can be processed and analyzed to determine respective concentrations of targeted analytes. In certain instances, the patch 14 includes one type of chemical indicator 18 (e.g., to help determine concentration of one type of analyte), but in other instances the patch 14 includes multiple types of chemical indicators.

For the implantable approach, the system 10 can include an implantable medical device 20, which includes one or more electrodes 22 and a chemical sensor assembly 24. The electrodes 22 can comprise a conductive material and be configured to sense cardiac activation signals. The chemical sensor assembly 24 can include a sensing element with a polymeric matrix permeable to analytes such as creatinine and/or potassium. The sensing element can include an interior volume with various chemical indicators (e.g., beads for detecting an ion concentration of a bodily fluid when implanted in the body disposed within an interior volume). Analytes can diffuse through an outer barrier layer and onto and/or into the chemical indicators where the analytes can bind with ion selective sensors to produce an optical response (e.g., a change in optical properties such as a change in concentration, a fluorimetric response, a colorimetric response). The optical response can be monitored and used to estimate analyte levels. The estimated analyte levels can be used by a computing device to monitor and evaluate a person's kidney and/or cardiac performance.

### Methods

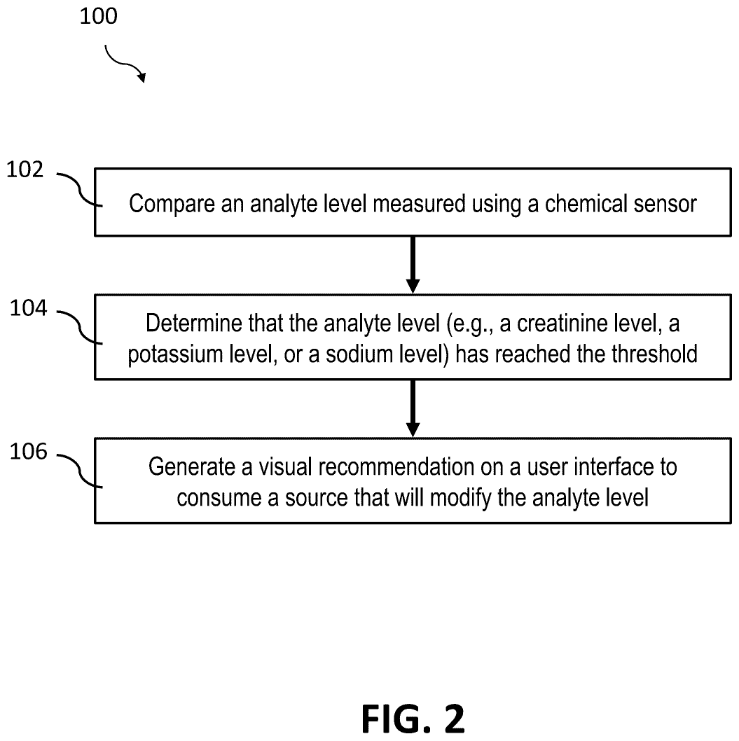

FIG. 2 outlines a method that can be used in connection with one or more components of the system 10 of FIG. 1 or other components described herein.

FIG. 2 shows a block diagram of a method 100 for making diet-related recommendations. In certain instances, the method 100 is carried out by one or more computing devices such as a mobile phone, a tablet, a laptop computer, a desktop computer, a server. For example, the method 100 could carried out by an application (e.g., a phone app) that is downloadable to the device, operated on the device, and accessible by a patient, their physician, their clinic, and the like.

The method 100 includes comparing an analyte level—measured using a chemical sensor (e.g., based on an optical property)—to a threshold (block 102 in FIG. 2). In certain instances, the analyte level being measured is a creatinine level, a potassium level, and/or a sodium level. The chemical sensor can be the types of chemical sensors described in connection with FIG. 1 and herein. A chemical indicator that is part of the chemical sensor can in communication with a person's blood such that one or more optical properties of the chemical indicator can be monitored to determine a level or concentration of the analyte. In certain instances, the analyte level is a current analyte level while in other instances, the analyte level is a future predicted analyte level (e.g., based on a person's historical analyte data such as their response to consuming a given type and amount of food).

In certain instances, comparing an analyte level to the threshold occurs periodically (e.g., every 30 minutes, once an hour) or on demand (e.g., when a patient or physician initiates the comparison). Although the chemical sensors may react in real-time (e.g., the chemical indicators change optical properties in real-time as analyte levels change in real-time), transmission of or calculating an analyte level and comparing the analyte level to the threshold less often can save computing and battery resources and may be preferable because analyte levels may not change drastically minute-by-minute.

The method 100 includes determining that the analyte level (e.g., a creatinine level, a potassium level, sodium level) has reached the threshold (block 104 in FIG. 2). As described below, different types of thresholds can be used.

When creatinine is being monitored, the threshold(s) can be set to identify when the patient has a creatinine level that is low enough for the patient to consume food such as food containing protein. Normal serum creatinine concentrations are between 0.6 and 1.3 mg/dL of blood. However, when renal function declines, less creatinine is excreted from the body, and serum concentrations of creatinine rise. A serum concentration of creatinine that is higher than 3.0 mg/dL is generally believed to indicate renal system failure. In some instances, the threshold is a predetermined level (e.g., 1.3 mg/dL of serum creatinine concentration) or a predetermined range (e.g., 1.5-3.0 mg/dL of serum creatinine concentration). In some instances, the threshold is a predetermined level below (e.g., 1-2 mg/dl of serum creatinine concentration) below historical analyte data (e.g., an average) or a predetermined percentage below (e.g., 10-25%) historical analyte data of the patient.

When potassium is being monitored, the thresholds can be set to identify when the patient has a potassium level that is low enough for the patient to consume food such as food containing potassium. Serum potassium (also known as K) measures the amount of potassium in the blood. Normal serum potassium levels for adults range from 3.5-5.0 mEq/L. In some instances, the threshold is a predetermined level (e.g., 3.5 mEq/L) or a predetermined range. In some instances, the threshold is a predetermined level below or above (e.g., 0.5) below historical analyte data (e.g., an average) or a predetermined percentage below or above (e.g., 10-25%) historical analyte data of the patient.

When sodium is being monitored, the thresholds can be set to identify when the patient has a sodium level that is low enough for the patient to consume food such as food containing sodium. Serum sodium measures the amount of potassium in the blood. Normal serum sodium levels for adults range from 136-145 mEq/L. In some instances, the threshold is a predetermined level (e.g., 136 and/or 145 mEq/L) or a predetermined range. In some instances, the threshold is a predetermined level below or above (e.g., 1) below historical analyte data (e.g., an average) or a predetermined percentage below or above (e.g., 10-25%) historical analyte data of the patient.

For certain analytes, analyte levels will naturally modulate over a 24-hour-day period. As such, the various thresholds described above can be time-dependent thresholds. For example, a given threshold might change depending on the time of day. The active threshold may be patient-unique (e.g., based on a person's historical analyte level variations) or set based on population-wide trends. Using potassium as an example, a person's typical potassium level might be 4.0 in the morning, 3.8 midday, 4.0 evening, and 4.2 at night. Active thresholds can be set such that a variance of +/−0.4 (or 0.5 or another number) from an expected potassium level at a given point in time will breach the threshold.

The method 100 further includes-after determining that the threshold has been reached-generating a visual recommendation on a user interface to consume a source that will change (e.g., increase) the analyte level or refrain from consuming a source (block 106 in FIG. 2).

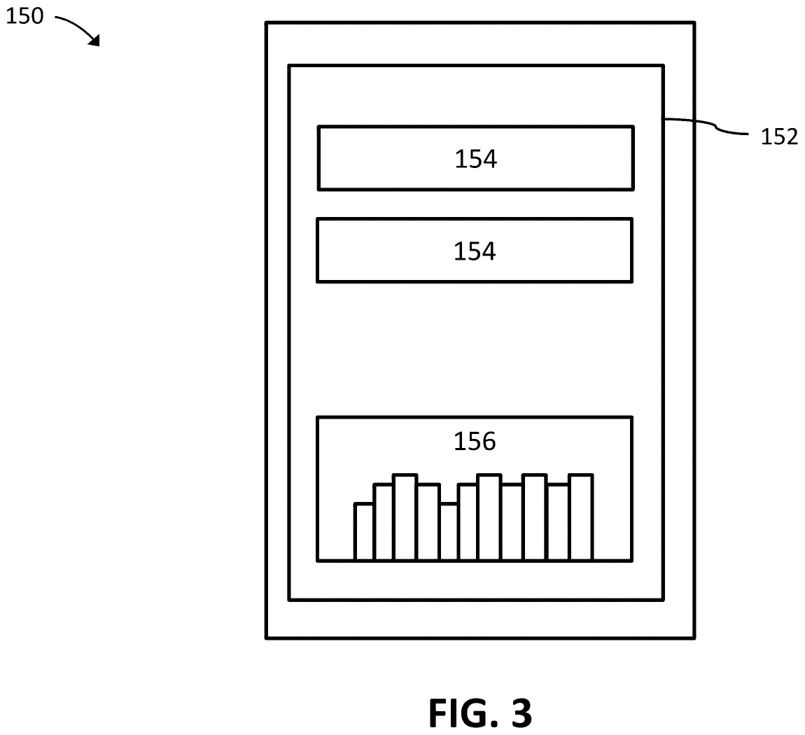

In certain embodiments, the visual recommendation can be displayed on a user interface of a computing device (e.g., a mobile computing device). FIG. 3 shows an example computing device 150 such as a mobile phone or tablet that includes a user interface 152. The device 150 can operate an application such that the user interface 152 displays various screens, icons, and/or buttons to help the user navigate and use the application. One or more graphics, pictures, videos, and/or text passages 154 can be used to for the visual recommendation to consume a source that will increase the analyte level (e.g., by consuming a source of protein or a source of potassium). In certain instances, the visual recommendation displays a suggested specific amount, an amount range, and/or a maximum amount of the source. The suggested amount, etc., can be in the form of a weight (e.g., ounces) or a portion size (e.g., small, medium, one fruit, one vegetable) of a particular type of food (e.g., chicken for protein, steak for protein, banana for potassium). As another example, the suggested amount, etc., can be in the form of an amount of protein (e.g., grams of protein) or potassium. Using this approach, a person can select a type of food that will meet the recommended amount and use, for example, nutritional information associated with the food to determine the proper serving size to reach the suggested amount.

The suggested amount calculated and then displayed as part of the visual recommendation can be based on the measured analyte levels and/or a prediction of future analyte levels based on historical data (e.g., a person's typical response to consuming the source). In certain instances, the visual recommendation displays a type of source (e.g., a type of food such as a picture of a chicken breast, a graphical representation of a banana, and the like).

When using a threshold associated with prior analyte data of the patient, one or more additional guardrails or thresholds can be used. For example, the method 100 can include comparing the analyte level to minimum threshold or maximum threshold before the graphical representation is generated or displayed. The threshold can be set such that—although a current (or predicted) analyte level is below or above historical levels—the current (or predicted) analyte level might still be too high or too low to recommend consuming food that will increase a person's analyte level. In such situations, the method 100 can prevent recommending that a person consume certain food if a current analyte level is above a minimum threshold or below a maximum threshold. Put another way, the method 100 can include recommending that a person consume certain food only if a current (or predicted) analyte level is below a minimum level or above a maximum level.

Any of the data described herein—whether historical or in real-time—can be displayed on the user interface 152 of the computing device 150. For example, the user interface 152 can be arranged to include a window 156 in which one or more sets of data can be displayed (e.g., levels as a function of time).

As noted above, a prediction of a person's future analyte level can be compared to a threshold to determine whether to generate a visual recommendation to consume a source of food.

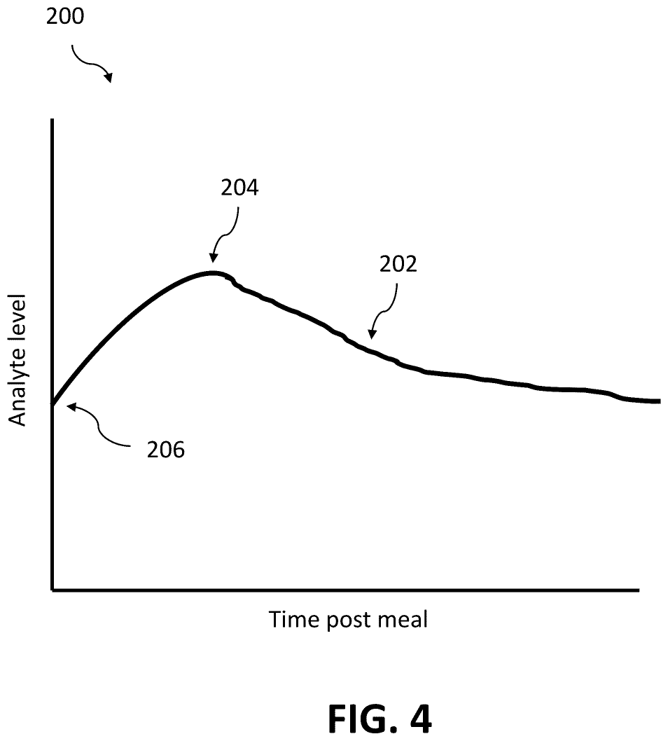

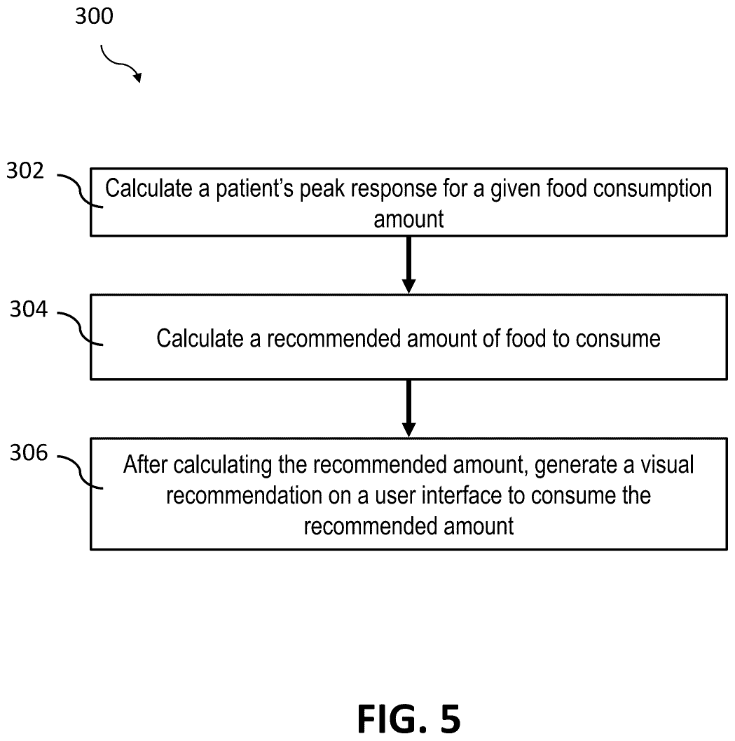

FIG. 4 shows a graph 200 of a person's response to consuming food, and FIG. 5 outlines a method that can be used in connection with a person's response to consuming food. The graph 200 includes a plot 202 of a person's creatinine level at a given time period after the person consumed food containing protein. Although creatinine is used as an example, similar approaches can be used with other analytes such as potassium. In the graph 200, the plot 202 includes a peak 204, which is the point in time at which the person's creatinine level rises the most after having consumed food.

A graph such as the graph 200 can be used for multiple purposes. As one example, graphs can be used to determine how drastic a person responds to consuming food. A large difference between the peak 204 and the pre-meal analyte level suggests that smaller portions should be recommended. As another example, graphs can be used to determine how long a meal increases a person's analyte levels. A long effect suggests recommending portions spaced farther away from each other.

FIG. 5 outlines a method that can be used in connection with one or more components of the system 10 of FIG. 1 or other components described herein.

FIG. 5 shows a block diagram of a method 300 for making diet-related recommendations based on a person's historical response to consuming food. In certain instances, the method 300 is carried out by one or more computing devices such as a mobile phone, a tablet, a laptop computer, a desktop computer, a server. For example, the method 300 could carried out by an application (e.g., a phone app) that is downloadable to the device, operated on the device, and accessible by a patient, their physician, their clinic, and the like.

The method 300 includes calculating a patient's peak response for a given food consumption amount (block 302 in FIG. 5). For example, the peak response can be the difference between the person's analyte level before food is consumed and the level at which the person's analyte level peaks after the food is consumed. In certain instances, the peak response is determined in response to a person consuming a known amount of food (e.g., a meal or food with a specific amount of protein or potassium).

The method 300 further includes calculating a recommended amount of food to consume (block 304 in FIG. 5). The recommended amount can be based, at least in part, on a safety threshold (e.g., a maximum level of analyte), the peak response calculated from the prior step of method 300. The safety threshold can be a predetermined level. The calculated recommended amount can be determined using the following equation:

Recommended amount=((Safety threshold−Cr_{Today})/(Peak response from block 302))*100 grams.

The method 300 further includes—after calculating the recommended amount—generating a visual recommendation on a user interface to consume the recommended amount (block 306 in FIG. 5). The visual recommendation can take the form of the other visual recommendations described herein.

### Wearable Chemical Sensor

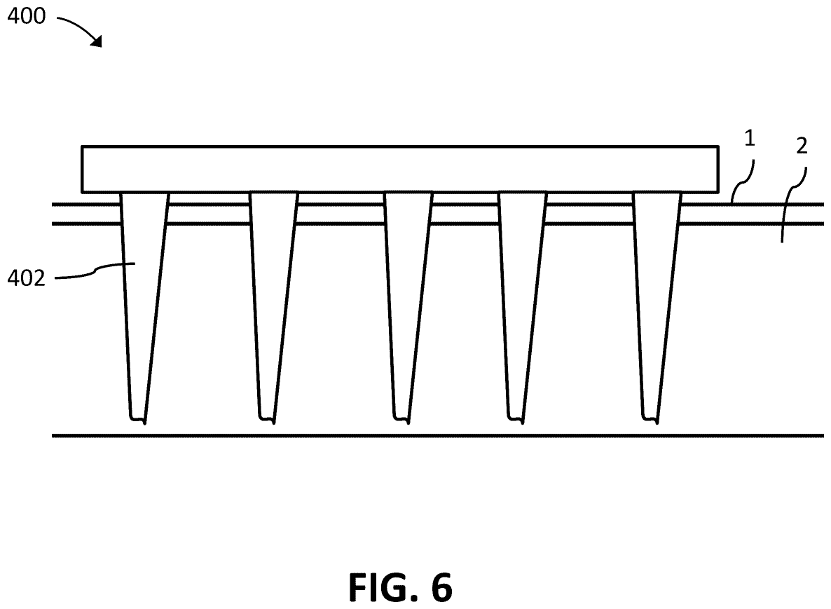

FIG. 6 shows a schematic side view of a wearable chemical sensing device 400. For simplicity, the device 400 is hereinafter referred to as the “patch 400” although other types of wearable devices can use the chemical sensing technology described herein. The patch 400 can be coupled to the patient's skin 1 such that needles 402 pierce through the outer layer of skin 1 and extend into the patient's interstitial fluid space 2. The needles 402 can have openings that are exposed to the patient's interstitial fluid (and therefore analytes within the patient's interstitial fluid).

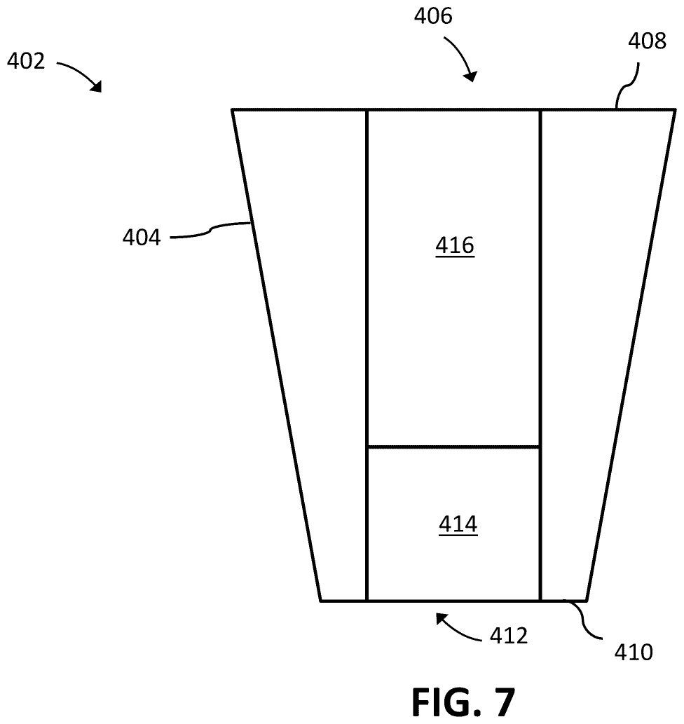

FIG. 7 shows a schematic side view of one of the needles 402 of the patch 400. In certain instances, the needles 402 are hollow needles such that each needle 402 includes an outer needle structure 404 that surrounds an opening 406 (e.g., a central thru-hole within the needle 402). The opening 406 can extend from a proximal end 408 of the needle 402 to a distal end 410 of the needle 402. An aperture 412 is located at or near the distal end 410 of the needle 402 such that the opening 406 is exposed to interstitial fluid.

Also at or near the distal end 410 of the needle 402 is a membrane 414 (e.g., a diffusion membrane) that is positioned within the needle 402. The membrane 414 protects tissue from direct interaction or exposure to a chemical indicator 416 that is also positioned within the needle 402. The membrane 414 can be formed from a permeable material, such as an ion permeable polymeric matrix material. In some instances, the membrane 414 can be permeable to sodium ions, potassium ions, hydronium ions, creatinine, urea, and various additional analytes. As referenced above, the cover membrane of the sensing element can be formed of a permeable material. In some embodiments, the cover membrane can be formed from an ion-permeable polymeric matrix material. Suitable polymers for use as the ion-permeable polymeric matrix material can include, but are not limited to, polymers forming a hydrogel. Hydrogels herein can include homopolymeric hydrogels, copolymeric hydrogels, and multipolymer interpenetrating polymeric hydrogels. Hydrogels herein can specifically include nonionic hydrogels. In certain instances, the membrane 414 includes an active agent disposed therein including, but not limited to anti-inflammatory agents, angiogenic agents, and the like.

The particular type (e.g., type of ion selectivity) and length of membrane can vary by needle 402. For example, one set of needles 402 can include a membrane 414 that is permeable to creatinine ions, while another set of needles 402 includes a membrane 414 that is permeable to potassium or sodium ions, and so on. In other examples, the membrane 414 is agnostic to a particular type of ion. The membrane 414 is positioned such that analytes must pass through the membrane 414 before reaching the chemical indicator 416. The membrane 414 material used will affect how fast an analyte travels between interstitial fluid and the chemical indicator 416.

The chemical indicator 416 comprises a material that changes properties (e.g., optical properties such as absorption, transmission, scattering, fluorescence) with changes in concentration of a given analyte. As one example, the chemical indicator 416 can comprise a creatinine select compound that changes optical properties in response to the creatinine select compound binding to creatine. As another example, the chemical indicator 416 can comprise a creatinine deiminase enzyme covalently bound to a substrate and a pH-indicating compound in ionic communication with the creatinine deiminase enzyme. In this example, the chemical indicator 416 can change optical properties in response to changes in creatinine concentrations in vivo. As another example, the chemical indicator 416 can comprise a creatinine select compound, and a pH-indicating compound in ionic communication with bodily fluid. In this example, the chemical indicator 416 can change optical properties in response to changes in creatinine concentrations in vivo. And in this example, the chemical indicator 416 may also comprise a mechanism to change local pH within the chemical indicator 416.

In certain instances, color of the chemical indicator 416 comprises the sum of the absorption, transmission, reflectance, and fluorescence properties of the chemical indicator material. Put another way, the chemical indicator 416 can comprise a material that changes optical properties with changes in concentration of a given analyte—and such optical properties can be measured by analyzing an image of the chemical indicator 416. In certain instances, the chemical indicator 416 has a minimum thickness or height along a longitudinal axis of a needle of 0.15-0.60 mm (e.g., 0.50-0.60 mm). In certain instances, the chemical indicator 416 comprises a slurry or a film.

In certain instances, the chemical indicator 416 is formed of a lipophilic indicator dye (e.g., a lipophilic fluorescent indicator dye or a lipophilic colorimetric indicator dye). Lipophilic indicator dyes can include, but are not limited to, ion selective sensors such as ionophores or fluorophores. In certain instances, ionophores can include sodium-specific ionophores, potassium-specific ionophores, calcium-specific ionophores, magnesium-specific ionophores, and lithium-specific ionophores. In certain instances, fluorophores can include lithium-specific fluorophores, sodium-specific fluorophores, and potassium-specific fluorophores.

Compositions of the chemical indicator 416 can include components (or response elements) that are configured for a colorimetric response, a photoluminescent response, or another optical sensing modality. For example, the chemical indicator 416 can include an element that changes color based on binding with or otherwise complexing with a specific chemical analyte. As one specific example, creatinine reacts with a molecule which changes pH and color on the indicator. In some instances, the chemical indicator 416 can include a complexing moiety and a colorimetric moiety. Those moieties can be a part of a single chemical compound (e.g., a non-carrier-based system) or can be separated on two or more different chemical compounds (e.g., a carrier-based system). The colorimetric moiety can exhibit differential light absorbance on binding of the complexing moiety to an analyte.

Some of the chemical indicators 416 may not require a separate compound to both complex an analyte of interest and produce an optical response. By way of example, in some instances, the response element can include a non-carrier optical moiety or material wherein selective complexation with the analyte of interest directly produces either a colorimetric or fluorescent response. As an example, a fluoroionophore can be used and is a compound including both a fluorescent moiety and an ion complexing moiety. As merely one example, (6,7-[2.2.2]-cryptando-3-[2″-(5″-carboethoxy)thiophenyl]coumarin, a potassium ion selective fluoroionophore, can be used (and in some cases covalently attached to polymeric matrix or membrane) to produce a fluorescence-based K+ non-carrier response element. An exemplary class of fluoroionophores are the coumarocryptands. Coumarocryptands can include lithium specific fluoroionophores, sodium specific fluoroionophores, and potassium specific fluoroionophores. For example, lithium specific fluoroionophores can include (6,7-[2.1.1]-cryptando-3-[2″-(5″-carboethoxy) furyl]coumarin. Sodium specific fluoroionophores can include (6,7-[2.2.1]-cryptando-3-[2″-(5″-carboethoxy) furyl]coumarin. Potassium specific fluoroionophores can include (6,7-[2.2.2]-cryptando-3-[2″-(5″-carboethoxy) furyl]coumarin and (6,7-[2.2.2]-cryptando-3-[2″-(5″-carboethoxy)thiophenyl]coumarin.

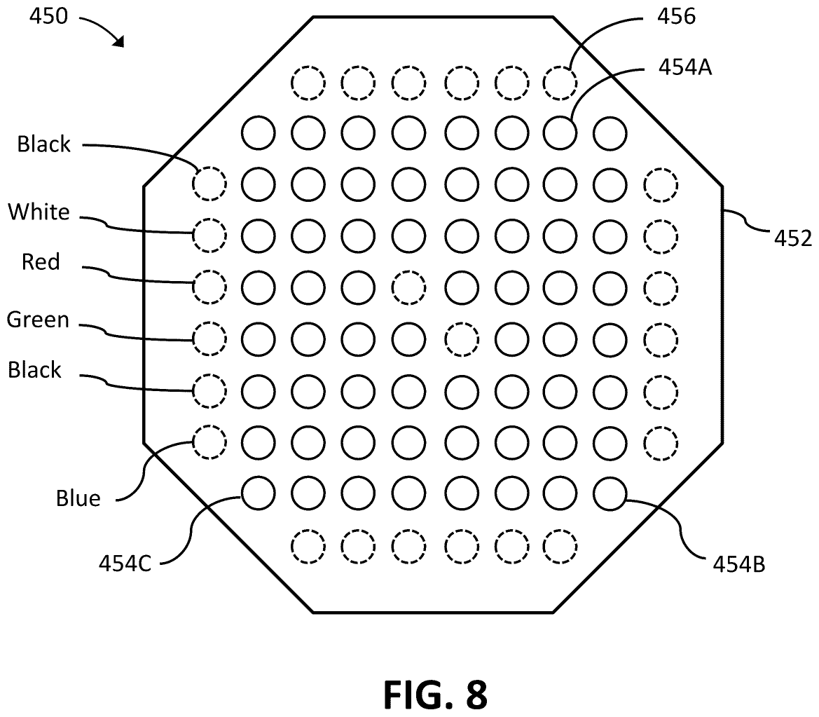

FIG. 8 shows a top view of a patch 450. The arrangement shown in FIG. 8 can be used in connections with the needles, membranes, chemical indicators, and layers, etc., described above with respect to the patch 400. The view shown in FIG. 8 is the type of view of a patch that an image sensor would capture in a digital image while the patch is coupled to the patient. The digital image can capture the colors of the chemical indicators (and color references) such that the colors can be analyzed to determine concentrations of one or more analytes of the patient.

FIG. 8 shows a patch 450, which includes a window 452 through which various components of the patch 100 can be viewed. In particular, the window 452 allows chemical indicators 454A-C of the patch 450 to be viewed. The chemical indicators 454A-C can be positioned in needles (e.g., hollow needles) such as the needles described above. As such, each chemical indicator 454A-C can be associated with its own needle.

A first set of needles can include a first type of chemical indicator 454A such as a chemical indicator that changes in color with changes in concentration of a first analyte (e.g., creatinine). A second set of needles can include a second type of chemical indicator sodium 454B such as a chemical indicator that changes in color with changes in concentration of a second analyte (e.g., potassium). A third set of needles can include a third type of chemical indicator 454C such as a chemical indicator that changes in color with changes in concentration of a third analyte (e.g., sodium). The respective colors of the chemical indicators can be used to estimate the respective concentrations of analytes in a patient's interstitial fluid.

In certain instances, each of the first type of chemical indicators 454A are positioned near or next to each other, each of the second type of chemical indicators 454B are positioned near or next to each other, and so on. The overall number of chemical indicators (and therefore the number of needles) and the number of different sets of types of chemical indicators on a given patch can be fewer or greater than that shown in FIG. 8. For example, the patch 450 could include a single type of chemical indicator selected for a single type of analyte. The relative positions of the chemical indicators 454A-C can vary from that shown in FIG. 8, and the specific shape of the chemical indicators 454A-C (as seen from a top view) can vary from the circular shapes shown in FIG. 8.

The patch 450 can also include color references 456. The color references 456 are shown in dotted lines in FIG. 8. The color references 456 can help with calibrating, correcting, and/or processing the digital image of the chemical indicators 454A-C such that an accurate estimate of the color of the chemical indicators 454A-C can be determined. For example, because the color of the color references 456 is known, the color of the chemical indicators 454A-C can be more accurately estimated as the patch 450 is positioned in different lighting (e.g., in direct sunlight, in a shadow, partially shaded, and the like). As such, the color references 456 can act as a color index or reference point for correcting for changes in color caused by ambient light.

In certain instances, some of the color references 456 are black, others white, others red, others green, others blue. Although most of the color references 456 in FIG. 8 are shown around a perimeter of the patch (e.g., with the chemical indicators 454A-C positioned within the perimeter), other positions and arrangements of the color references 456 can be utilized in the patch 450. The overall number and the specific shape of the color references 456 (as seen from a top view) can vary from the circular or dot shape shown in FIG. 8.

Using the patches described herein, analyte concentrations can be estimated. For example, a digital image of a patch attached to a patient can be taken by a camera and an analyte concentration can be estimated based on a color of one or more chemical indicators. In certain instances, estimating the analyte concentrations involves calculating an analyte concentration for multiple chemical indicators and then applying a mathematical operation (e.g., averaging, voting) to determine the respective analyte concentrations. The analyte concentration estimations can be further based on corrections that are determined using color reference sections of the patch. Each set or grouping of chemical indicators from the digital image can be processed and their respective colors compared to a table, library, mapping, index, etc. that associates a given color of chemical indicator to a given concentration level. In certain instances, the process of estimating analyte concentrations is carried out by an application stored on and operated by a smart phone. In other instances, some or all steps can be carried out by a server or other computing system besides a smart phone that can access digital images of a patch and be programmed to determine estimated analyte concentration levels based on colors of chemical indicators shown in the digital image.

U.S. patent application Ser. No. 18/774,681 describes additional details of a wearable chemical sensing system and is herein incorporated by reference in its entirety.

### Implantable Chemical Sensor

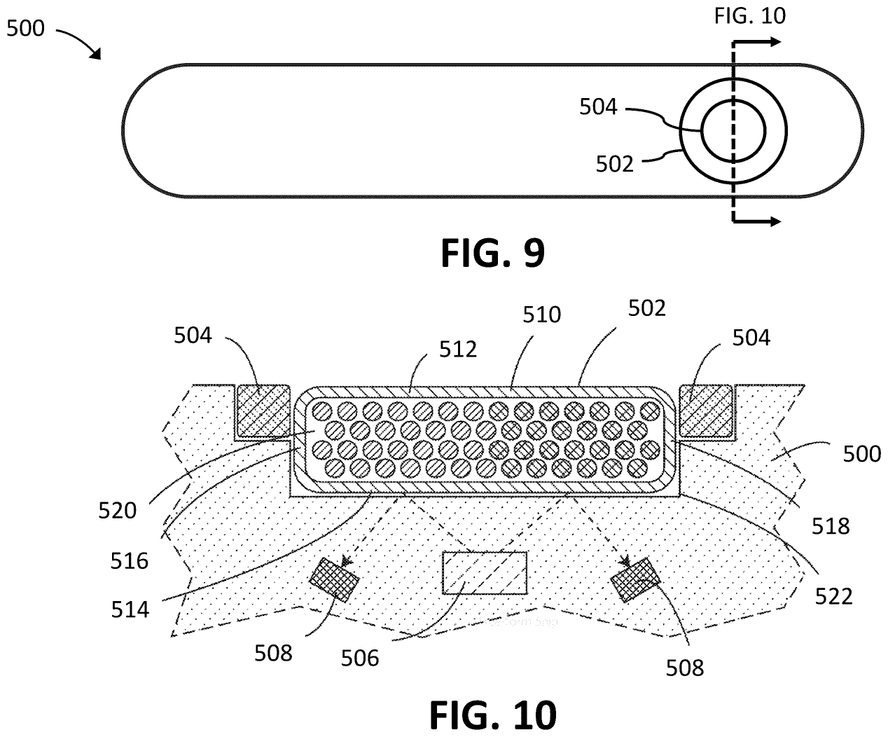

FIGS. 9 and 10 show an implantable medical device (IMD) 500 with a chemical sensor assembly.

FIG. 9 shows a top-down view of the IMD 500 with a sensing element 502 and an active agent eluting material 504 (or active agent eluting matrix) disposed around the outer perimeter of sensing element 502. In some instances, the active agent eluting material 504 forms a ring structure around the outer perimeter of sensing element 502. It will be appreciated that the sensing element 502 and the active agent eluting material 504 can have different geometric shapes and sizes.

FIG. 10 shows a cross-sectional view of a portion of the IMD 500 and its chemical sensor assembly. The IMD 500 includes an optical excitation assembly 506 and optical detection assembly 508. The sensing element 502 can include an outer barrier layer 510 formed, in full or in part, from a permeable material, such as an ion permeable polymeric matrix material. The outer barrier layer 510 can form a top 512, a bottom 514, and opposed sides 516 and 518 to surround an interior volume 520 of the sensing element 502. In certain instances, at least the top 512 of outer barrier layer 510 is permeable to sodium ions, potassium ions, hydronium ions, creatinine, urea, and the like. The outer barrier layer 510 can also include an active agent disposed therein including, but not limited to anti-inflammatory agents, angiogenic agents, and the like. The housing of the IMD 500 can include a recessed pan 522 into which the sensing element 502 fits. In some embodiments, implantable housing can define an aperture occluded by a transparent member. The transparent member can be a glass (including but not limited to borosilicate glasses), a polymer or other transparent material. The aperture can be disposed at the bottom of the recessed pan. The aperture can provide an interface allowing for optical communication between sensing element and the optical excitation and optical detection assemblies. It will be appreciated that outer barrier layer, or portions thereof, can be made from a transparent polymer matrix material to allow for optical communication between the sensing element 502 and optical excitation 506 and optical detection 508 assemblies.

The optical excitation assembly 506 can be designed to illuminate the sensing element 502. The optical excitation assembly 508 can include a light source such as a light emitting diode (LED), vertical-cavity surface-emitting lasers (VCSELs), electroluminescent (EL) devices, and the like. The optical detection assembly 508 can include a component selected from the group consisting of a photodiode, a phototransistor, a charge-coupled device (CCD), a junction field effect transistor (JFET) optical sensor, a complementary metal-oxide semiconductor (CMOS) optical sensor, an integrated photo detector integrated circuit, a light to voltage converter, and the like.

Various indicator beads can be positioned in the interior volume 520. The indicator beads can be used for detecting an ion concentration of a bodily fluid. For example, the indicator beads can include a polymeric support material and one or more ion selective sensing components as described more fully below. Analytes such as creatinine, potassium ion, sodium ion, hydronium ion, and the like, can diffuse through the top of the outer barrier layer and onto and/or into the indicator beads where they can bind with the ion selective sensors to produce a change in optical properties (e.g., a fluorimetric response, a colorimetric response).

U.S. Patent App. Pub. No. 2018/0344218 describes additional details of an implantable medical device with a chemical sensor assembly and is herein incorporated by reference in its entirety.

### Computing Device

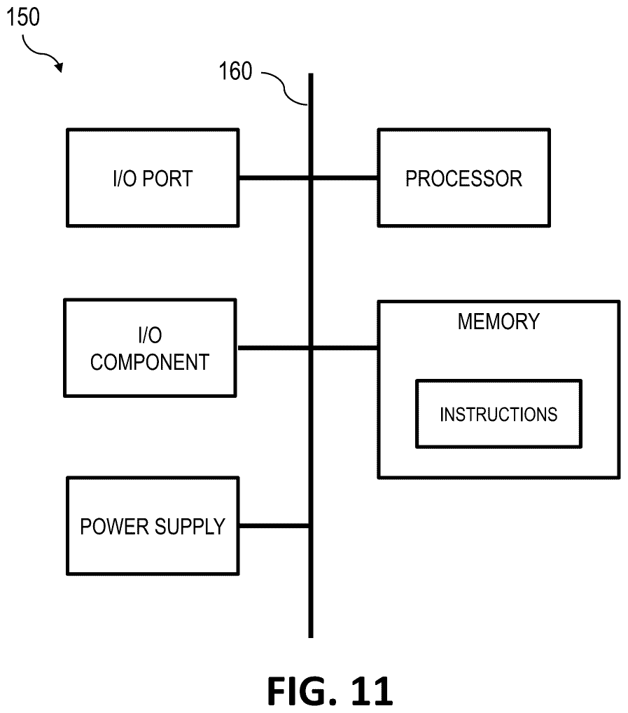

FIG. 11 is a block diagram depicting additional details of the computing device 150 shown in FIG. 3. The computing device 150 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like.

In instances, the computing device 150 includes a bus 160 that, directly and/or indirectly, couples one or more of the following devices: a processor, a memory, an input/output (I/O) port, an I/O component, and a power supply. Any number of additional components, different components, and/or combinations of components may also be included in the computing device 150.

The bus 160 represents what may be one or more busses (such as, for example, an address bus, data bus, or combination thereof). Similarly, in instances, the computing device 150 may include a number of processors, a number of memory components, a number of I/O ports, a number of I/O components, and/or a number of power supplies. Additionally, any number of these components, or combinations thereof, may be distributed and/or duplicated across a number of computing devices.

In instances, the memory includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory stores computer-executable instructions for causing the processor to implement aspects of instances of components discussed herein and/or to perform aspects of instances of methods and procedures discussed herein. The memory can comprise a non-transitory computer readable medium storing the computer-executable instructions.

The computer-executable instructions may include, for example, computer code, machine-useable instructions, and the like such as, for example, program components capable of being executed by one or more processors (e.g., microprocessors) associated with the computing device 150. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions may be configured to be executed by the processor and, upon execution, to cause the processor to perform certain processes. In certain instances, the processor, memory, and instructions are part of a controller such as an application specific integrated circuit (ASIC), field-programmable gate array (FPGA), and/or the like. Such devices can be used to carry out the functions and steps described herein.

The I/O component may include a presentation component configured to present information to a user such as, for example, a display device, a speaker, a printing device, and/or the like, and/or an input component such as, for example, a microphone, a joystick, a satellite dish, a scanner, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A system comprising:
a mobile computing device including a processor, memory, and a user interface, wherein the mobile computing device is programmed to:
compare an analyte level measured using a chemical sensor and based on an optical property to a threshold, wherein the analyte level is a creatinine level, a potassium level, or a sodium level,
determine that the creatinine level, the potassium level, or the sodium level has reached the threshold, and
in response, display a visual recommendation on the user interface to consume a source of protein, a source of potassium, or a source of sodium.

2. The system of claim 1, further comprising: the chemical sensor, wherein the chemical sensor is a wearable device with a first set of needles sized for access to interstitial fluid and a first chemical indicator positioned within the first set of needles, wherein the first chemical indicator changes optical properties in response to different creatinine levels.

3. The system of claim 2, wherein the chemical sensor includes a second set of needles sized for access to interstitial fluid and a second chemical indicator positioned within the second set of needles, wherein the second chemical indicator changes optical properties in response to different potassium levels.

4. The system of claim 2, wherein the chemical sensor includes a second set of needles sized for access to interstitial fluid and a second chemical indicator positioned within the second set of needles, wherein the second chemical indicator changes optical properties in response to different sodium levels.

5. The system of claim 2, wherein the mobile computing device includes an image sensor.

6. The system of claim 5, wherein the mobile computing device is programmed to:
determine the creatinine level based, at least in part, on the optical properties.

7. The system of claim 1, wherein the mobile computing device includes an image sensor and is programmed to: determine the creatinine level, the potassium level, or the sodium level based, at least in part, on an optical property of the chemical indicator in a digital image.

8. The system of claim 1, further comprising: the chemical sensor, wherein the chemical sensor is part of an implantable medical device and comprises a chemical indicator, wherein the chemical indicator changes optical properties in response to different creatine levels, potassium levels, or sodium levels.

9. The system of claim 1, wherein the visual recommendation displays an amount or a range of the source.

10. The system of claim 1, wherein the visual recommendation displays a type of source.

11. The system of claim 1, wherein the mobile computing device is programmed to: generate the visual recommendation only if the creatine level, the potassium level, or the sodium level is below a minimum level.

12. The system of claim 1, wherein the threshold is a predetermined percentage below a predetermined level or below historical analyte data.

13. The system of claim 1, wherein the threshold is a predetermined level below a predetermined level or below historical analyte data.

14. A method comprising:
comparing an analyte level measured using a chemical sensor and based on an optical property to a threshold, wherein the analyte level is a creatinine level, a potassium level, or a sodium level;
determining that the creatinine level, the potassium level, or the sodium level has reached the threshold; and
after the determining, generating a visual recommendation on a user interface to consume or refrain from consuming a source of protein, a source of potassium, or a source of sodium.

15. The method of claim 14, further comprising: determining the creatinine level, the potassium level, or the sodium level based, at least in part, on the optical property contained in a digital image taken by an image sensor.

16. The method of claim 14, wherein the visual recommendation displays an amount or a range of the source.

17. The method of claim 14, wherein the visual recommendation displays a type of source.

18. The method of claim 14, further comprising: generating the visual recommendation only if the creatine level, the potassium level, or the sodium level is below a minimum level.

19. The method of claim 14, wherein the threshold is a predetermined percentage below a predetermined level or below historical analyte data.

20. The method of claim 14, wherein the threshold is a predetermined level below a predetermined level or below historical analyte data.
